(19) United States
(12) Patent Application Publication
(10) Pub. No.: US 2008/0242911 A1
(43) Pub. Date: Oct. 2, 2008

(54) CH ACTIVATION/DEHYDROGENATION OF HYDROCARBONS

(75) Inventors: Ingrid Boehmer, Forchheim (DE); M. Bruce Welch, Bartlesville, OK (US); Roland Schmidt, Bartlesville, OK (US); Bruce B. Randolph, Bartlesville, OK (US); Helmut G. Alt, Bayreuth (DE)

Correspondence Address: ConocoPhillips Company;I. P. Legal, P.O. Box 2443, Bartlesville, OK, 74005, US

(73) Assignee: ConocoPhillips Company, Houston, TX (US)

(21) Appl. No.: 11/865,882

(22) Filed: Oct. 2, 2007

Related U.S. Application Data
(62) Division of application No. 10/866,158, filed on Jun. 12, 2004, now abandoned.

(51) Int. Cl. C07C 5/333

(52) U.S. Cl. 585/660; 585/661

(57) ABSTRACT

A catalyst comprising a
1) complex comprising:
   a) at least one metal selected from the group consisting of Group 8 metals, Group 9 metals, Group 10 metals, and combinations thereof;
   b) a compound having the formula R₃X wherein R is selected from the group consisting of hydrogen, an alkyl, an alkenyl, an alkynyl, cycloalkyls, cycloalkenyls, cycloalkynyls, aryls, substituted aryls, and substituted organic compounds and wherein X is a Group 15 element selected from the group consisting of nitrogen, phosphorus, arsenic, antimony and bismuth; and
2) support component comprising a silicon-containing compound and a method of making said catalyst, is disclosed.

The catalyst is then used to dehydrogenate hydrocarbons in a dehydrogenation reaction zone under dehydrogenation reaction conditions.

This invention relates to CH activation of hydrocarbon feedstocks. In another aspect, this invention relates to compositions suitable for use in the CH activation reactions of hydrocarbon feedstocks. A further aspect of this invention relates to processes for the production of compositions for use in the CH activation reactions of hydrocarbon feedstocks.

### BACKGROUND OF THE INVENTION

One of the most attractive goals in petrochemical research is to find compositions that are able to activate saturated hydrocarbons. The CH activation reaction involves the splitting of carbon-hydrogen bonds in hydrocarbons to form derivatives of the original hydrocarbon with at least two fewer hydrogen atoms than the original hydrocarbon. One form of CH activation is dehydrogenation, which is the catalytic reaction of alkanes and other dehydrogenable hydrocarbons to form cyclics, monoolefins, diolefins, and other compounds containing the same number of carbon atoms but fewer hydrogen atoms. The ubiquitous nature of saturated CH bonds implies that the ability to convert aliphatic CH bonds into other functional groups would significantly multiply the potential uses of hydrocarbon feedstocks and their commercial values. In contrast to the polymerization process, the dehydrogenation of alkanes is endothermic. Energy has to be supplied into the system, e.g., thermally or photochemically. Catalysts can reduce the activation energy of the reaction, but they do not alter the energy content of the reactants or products.

The key step of catalytically induced CH activation reactions is the formation of an electronically and coordinatively unsaturated species to enable an oxidative addition of an alkane to the metal center. In a second step, beta-hydrogen elimination can be induced thermally or photochemically to produce the olefin and hydrogen in the catalytic cycle. The use of organo-metallic compounds offers the possibility to influence the properties of the metal center by various ligands. These ligands influence the properties of the metal center in a way that beta-hydrogen elimination and simultaneous formation of the alkene ligand are facilitated. Consequently, finding a composition with a dynamic ligand system would be a significant contribution to the art and to the economy.

### SUMMARY OF THE INVENTION

It is thus an object of the present invention to provide a novel composition.

It is yet another object of the present invention to provide a process for the CH activation of hydrocarbon feedstocks.

In accordance with the present invention, the inventive composition comprises, consists of, or consists essentially of:
a) a complex comprising, consisting of, or consisting essentially of:
   i) at least one metal selected from the group consisting of Group 8 metals, Group 9 metals, Group 10 metals, and combinations thereof; and
   ii) a compound having the formula R₃X wherein R is selected from the group consisting of hydrogen, an alkyl, an alkenyl, an alkynyl, cycloalkyls, cycloalkenyls, cycloalkynyls, aryls, substituted aryls, and substituted organic compounds and wherein X is a Group 15 element selected from the group consisting of nitrogen, phosphorus, arsenic, antimony and bismuth; and
b) a support component comprising a silicon-containing compound
   wherein at a temperature in the range of from about 300° C. to about 650° C., and in the presence of a medium conducive to forming the composition, said composition has a carbon to Group 15 element mole ratio of from about 0.01:1 to about 18:1.

The second embodiment of the present invention includes a novel process comprising, consisting of, or consisting essentially of:

contacting a hydrocarbon feed with a catalyst in a dehydrogenation reaction zone under dehydrogenation reaction conditions wherein the catalyst at a temperature range of from about 0° C. to about 400° C., comprises:
a) a complex comprising
   (i) at least one metal selected from the group consisting of Group 8 metals, Group 9 metals, Group 10 metals, and combinations thereof; and
   (ii) a compound having the formula R₃X, wherein R is selected from the group consisting of hydrogen, an alkyl, an alkenyl, an alkynyl, cycloalkyls, cycloalkenyls, cycloalkynyls, aryls, substituted aryls, and substituted organic compounds and X is a Group 15 element selected from the group consisting of nitrogen, phosphorus, arsenic, antimony and bismuth; and
b) a support component comprising a silicon-containing compound.

The third and fourth embodiments of the present invention involve preparation methods for the composition in the first embodiment and the catalyst in the second embodiment.

The third embodiment is a method comprising, consisting of, or consisting essentially of:
a) admixing
   1) a liquid and
   2) a complex comprising
      i) a compound having the formula R₃X, wherein R is selected from the group consisting of hydrogen, an alkyl, an alkenyl, an alkynyl, cycloalkyls, cycloalkenyls, cycloalkynyls, aryls, substituted aryls, and substituted organic compounds and X is a Group 15 element selected from the group consisting of nitrogen, phosphorus, arsenic, antimony and bismuth; and
      ii) at least one metal selected from the group consisting of Group 8 metals, Group 9 metals, Group 10 metals, and combinations thereof, and
b) incorporating the mixture into or onto a silicon-containing compound.

The fourth embodiment is a method comprising, consisting of, or consisting essentially of:

a) incorporating at least one compound comprising at least one metal selected from the group consisting of Group 8 metals, Group 9 metals, Group 10 metals, and combinations thereof into or onto a silicon-containing compound to form a first incorporated mixture;

b) drying said first incorporated mixture to form a first dried incorporated mixture;

c) incorporating at least one compound having the formula R₃X wherein R is selected from the group consisting of hydrogen, an alkyl, an alkenyl, an alkynyl, cycloalkyls, cycloalkenyls, cycloalkynyls, aryls, substituted aryls, and substituted cyclic organic compounds and wherein X is a Group 15 element selected from the group consisting of nitrogen, phosphorus, arsenic, antimony and bismuth; into or onto said first dried incorporated mixture so as to form a second incorporated mixture; and

d) drying said second incorporated mixture to form said catalyst.

Other aspects, objectives, and advantages of the present invention will be apparent from the detailed description of the invention and the appended claims.

### DETAILED DESCRIPTION OF THE INVENTION

In accordance with the present invention, the inventive composition comprises, consists of, or consists essentially of:
a) a complex comprising, consisting of, or consisting essentially of:
   i) at least one metal selected from the group consisting of Group 8 metals, Group 9 metals, Group 10 metals, and combinations thereof; and
   ii) a compound having the formula R₃X wherein R is selected from the group consisting of hydrogen, an alkyl, an alkenyl, an alkynyl, cycloalkyls, cycloalkenyls, cycloalkynyls, aryls, substituted aryls, and substituted organic compounds and wherein X is a Group 15 element selected from the group consisting of nitrogen, phosphorus, arsenic, antimony and bismuth; and
b) a support component comprising a silicon-containing compound
   wherein at a temperature in the range of from about 300° C. to about 650° C., and in the presence of a medium conducive to forming said composition, the composition has a carbon to Group 15 element mole ratio of from about 0.01:1 to about 18:1.

In accordance with the present invention, the second embodiment of the present invention comprises, consists of, or consists essentially of:

contacting a hydrocarbon feed with a catalyst in a dehydrogenation reaction zone under dehydrogenation reaction conditions wherein the catalyst at a temperature range of from about 0° C. to about 400° C. comprises, consists of, or consists essentially of:

a) a complex comprising, consisting of, or consisting essentially of
(i) at least one metal selected from the group consisting of Group 8 metals, Group 9 metals, Group 10 metals, and combinations thereof; and
(ii) a compound having the formula R₃X wherein R is selected from the group consisting of hydrogen, an alkyl, an alkenyl, an alkynyl, cycloalkyls, cycloalkenyls, cycloalkynyls, aryls, substituted aryls, and substituted organic compounds and X is a Group 15 element selected from the group consisting of nitrogen, phosphorus, arsenic, antimony and bismuth; and

b) a support component comprising a silicon-containing compound.

The Periodic Table referred to in this application is the IUPAC Periodic Table of the Elements.

The inventive composition and the catalyst employed in the inventive process comprises a complex containing at least one metal selected from the group consisting of Group 8 metals, Group 9 metals, Group 10 metals, and combinations thereof. A complex is defined as the species formed by the Lewis acid-base reaction of a metal atom or ion with ligands.

The at least one Group 8, 9 or 10 metal can be selected from the group consisting of iridium, rhodium, platinum, nickel, cobalt, palladium, iron, ruthenium, osmium, and combinations of any two or more thereof. Preferably, the metal is iridium or platinum.

Generally, the metal is present in the catalyst composition in a weight percent in the range of from about 0.01 to about 10 weight percent, preferably in the range of from about 0.1 to about 5 weight percent and most preferably in the range of from 0.2 to 2 weight percent based on the total weight of the catalyst composition.

Any suitable compound having the formula R₃X can be used in the process of the present invention. Generally, “R” can be selected from the group consisting of hydrogen, an alkyl, an alkenyl, an alkynyl, cycloalkyls, cycloalkenyls, cycloalkynyls, aryls, substituted aryls, and substituted organic compounds. Generally, “X” is a Group 15 element selected from the group consisting of nitrogen, phosphorus, antimony, and bismuth. Preferably, the, Group 15 element is phosphorus.

Preferably, the compound is an organophosphine. Preferred organophosphines include, but are not limited to, triphenylphosphine and tricyclohexylphosphine. The organophosphine can be a part of an organophosphine-containing compound. The R₃X compound can bind to the metal and can become part of the complex.

The catalyst also includes a support component comprising a silicon-containing compound. Any suitable silicon containing material may be employed in the catalyst such as, for example, silica, diatomite, colloidal silica, silica gel, precipitated silica, and the like, and combinations thereof. In addition, silicon compounds that are convertible to silica can also be employed. Other silicon-containing compounds can be used, such as, for example, silicon carbide and silicon nitride. Most preferably, the support component is silica. The silicon-containing compound can either be dried or undried. Preferably, the silicon-containing compound is undried.

Preferably, the silicon-containing compound used in the inventive production method has a pore volume in the range of from about 0.01 cm³/g to about 10 cm³/g and a surface area in the range of from about 10 m²/g to about 1000 m²/g.

Generally, at a temperature in the range of from about 300° C. to about 650° C., and in the presence of a medium conducive to forming the composition, the inventive composition has a carbon to Group 15 element mole ratio of from about 0.01:1 to about 18:1. Preferably, the carbon to Group 15 element mole ratio is in the range of from about 0.01:1 to about 14:1. Most preferably, the carbon to Group 15 element mole ratio is in the range of from 0.01:1 to 10:1. Any medium conducive to forming the inventive composition can be used. Such mediums include, but are not limited to, nitrogen, a Group 18 element, hydrogen, a vacuum, hydrocarbons, and combinations thereof.

The inventive composition and the catalyst employed in the inventive process can be prepared by a method comprising, consisting of, or consisting essentially of:
a) admixing
   1) a liquid and
   2) a complex comprising
      i) a compound having the formula R₃X wherein R is selected from the group consisting of hydrogen, alkyl, an alkenyl, an alkynyl, cycloalkyls, cycloalkenyls, cycloalkynyls, aryls, substituted aryls, and substituted organic compounds, and X is a Group 15 element selected from the group consisting of nitrogen, phosphorus, arsenic, antimony and bismuth.
      ii) at least one metal selected from the group consisting of Group 8 metals, Group 9 metals, Group 10 metals, and combinations to form a mixture thereof, and
b) incorporating the mixture into or onto a silicon-containing compound.

In order to obtain the inventive composition, the recovered compounds from the inventive preparation methods must be heated to a temperature in the range of from about 300° C. to about 650° C. in the presence of a medium conducive to forming the inventive composition.

In the inventive process, the catalyst can generally be prepared by admixing a liquid and a complex comprising at least one compound having the formula R₃X and at least one metal selected from the group consisting of Group 8 metals, Group 9 metals, Group 10 metals, and combinations thereof to form a mixture thereof. The term “admixing” as used herein, denotes mixing components in any order and/or any combination or sub-combination. Any suitable means for admixing the components can be used to achieve the desired dispersion of such components. Examples of suitable admixing include, but are not limited to, mixing tumblers, stationary shelves or troughs, Euro Star mixers, which are of the batch or continuous type, impact mixers, magnetic stirrers, mechanical stirrers, and the like.

The liquid can be any solvent capable of dispersing and/or dissolving a complex comprising at least one compound having the formula R₃X and at least one metal selected from the group consisting of Group 8 metals, Group 9 metals, Group 10 metals, and combinations thereof. Preferably, the liquid can be selected from the group consisting of water, light hydrocarbons, aromatics, alcohols, acetone, toluene and halogenated hydrocarbons. More preferably, the liquid is toluene or dichloromethane.

Any suitable compound having the formula R₃X can be used in the preparation of the catalyst for the inventive process. R is generally selected from the group consisting of hydrogen, an alkyl, an alkenyl, an alkynyl, cycloalkyls, cycloalkenyls, cycloalkynyls, aryls, substituted aryls, and substituted organic compounds. X is generally selected from the group consisting of nitrogen, phosphorus, arsenic, antimony, and bismuth.

Preferably, the compound is an organophosphine. Preferably, it is in the form of an organophosphine or in the form of one or more organophosphine-containing compounds. Preferably, the organophosphine is in the form of triphenylphosphine or tricyclohexylphosphine.

The mixture is added to the silicon-containing compound by means of incorporation.

A preferred method of incorporating is to impregnate using any conventional incipient wetness impregnation technique (i.e., essentially completely or partially filling the pores of substrate material with a solution of the incorporating elements) for impregnating a substrate. This preferred method uses an impregnating solution comprising the desirable concentration of the complex to ultimately provide the catalyst used in the inventive process. The amount of liquid that can be absorbed by the silicon-containing compound is determined by the following method:

To one-gram of the silicon-containing compound, the solvent is added drop wise until the liquid becomes visible around the particles. The required amount of solvent can be calculated by the weight difference. The complex is dissolved in exactly the amount of a suitable solvent that is required to fill all pores of the support. The solution is then added drop wise to the silicon-containing compound and then dried in a nitrogen stream, heat and/or under a vacuum.

If a single-step impregnation is not possible, then the process can be completed in several steps. The complex can be added to the solvent, the solvent is then added to the silicon-containing compound via incipient wetness, as described above, and the resulting substance is then dried. Then the process can be repeated until the desired amount of the complex is added.

To obtain the inventive composition, the compound must be heated to a temperature in the range of from about 300° C. to about 650° C.

The inventive composition and the catalyst employed in the inventive process can also be prepared by a method comprising, consisting of, or consisting essentially of:

a) incorporating a compound comprising at least one metal selected from the group consisting of Group 8 metals, Group 9 metals, Group 10 metals, and combinations thereof into or onto a silicon-containing compound to form a first incorporated mixture;

b) drying said first incorporated mixture to form a first dried incorporated mixture;

c) incorporating at least one compound having the formula R₃X wherein R is selected from the group consisting of hydrogen, an alkyl, an alkenyl, an alkynyl, cycloalkyls, cycloalkenyls, cycloalkynyls, aryls, substituted aryls, and substituted cyclic organic compounds and wherein X is a Group 15 element selected from the group consisting of nitrogen, phosphorus, arsenic, antimony and bismuth; into or onto the first dried incorporated mixture so as to form a second incorporated mixture; and

d) drying the second incorporated mixture to form a dried second incorporated mixture and,

e) heating said dried second incorporated mixture at a temperature of from about 300° C. to about 650° C. in the presence of a medium conducive to forming said composition.

In the inventive process, the catalyst can generally be prepared by admixing a liquid and a compound comprising at least one metal selected from the group consisting of Group 8 metals, Group 9 metals, Group 10 metals, and combinations thereof to form a first mixture and then incorporating the first mixture into or onto a silicon-containing compound to form a first incorporated mixture. This is then dried to form a dried first incorporated mixture. Then, a second mixture is formed by admixing a liquid and a compound having the formula R₃X. The second mixture is then incorporated into or onto the dried first incorporated mixture. This resulting second incorporated mixture is then dried.

The metal compound can be incorporated onto the silicon-containing compound before, or after, or at the same time as the R₃X compound.

The liquid can be any solvent capable of dispersing a compound comprising at least one metal selected from the group consisting of Group 8 metals, Group 9 metals, Group 10 metals, and combinations thereof and a compound having the formula R₃X. Preferably, the liquid can be selected from the group consisting of water, light hydrocarbons, aromatics, alcohols, acetone, toluene and halogenated hydrocarbons. Most preferably, the liquid is toluene.

Both the first mixture and the second mixture are incorporated into or onto the silicon-containing compound and to the dried first incorporated mixture, respectively.

A preferred method of incorporating is to impregnate using any conventional incipient wetness impregnation technique (i.e., essentially completely or partially filling the pores of substrate material with a solution of the incorporating elements) for impregnating a substrate. This preferred method uses an impregnating solution comprising the desirable concentration of the compound comprising at least one metal selected from the group consisting of Group 8 metals, Group 9 metals, Group 10 metals, and combinations thereof or R₃X compound to ultimately provide the catalyst used in the inventive process. The amount of liquid that can be absorbed by the silicon-containing compound is determined by the following method:

To the one-gram of silicon-containing compound, the solvent is added drop wise until the liquid was visible around the particles. The required amount of solvent can be calculated by the weight difference. The compound comprising at least one metal selected from the group consisting of Group 8 metals, Group 9 metals, Group 10 metals, and combinations thereof or R₃X compound is dissolved in exactly the amount of a suitable solvent that is required to fill all pores of the support. The solution is then added drop wise to the silicon-containing compound or the dried first incorporated mixture and then dried in a nitrogen stream, heat and/or a vacuum.

In carrying out the inventive process, the dehydrogenation reaction conditions in the dehydrogenation reaction zone comprise a reaction temperature in the range of from about 150° C. to about 1000° C. Preferably the dehydrogenation reaction conditions include a reaction temperature in the range of from about 200° C. to about 650° C. and, most preferably, the dehydrogenation reaction conditions comprise a reaction temperature in the range of from 350° C. to 600° C. The hydrocarbon feed suitable for the inventive process is any hydrocarbon feed that can be dehydrogenated. Examples include, but are not limited to, alkanes with 2 to 10 carbon atoms per molecule. Preferably, the hydrocarbon feed is normal pentane, isopentane, cyclopentane, or combinations thereof. The catalyst can be reactivated by stripping with hydrogen.

The following examples are presented to further illustrate the invention and are not to be considered as limiting the scope of the invention.

### EXAMPLES

### Example I

Undried silica was impregnated with 99 milligrams of hydridocarbonyltris(triphenylphosphine)iridium(I) by incipient wetness. The iridium complex was dissolved in 11.53 grams of toluene. This solution was then added drop wise to 6.005 grams of silica granules (20×40 mesh), with a surface area of 315 m²/g and a pore volume of 1.1 cc/g, and was dried at about 50° C. and with a purge of nitrogen.

A 5.015-gram quantity of the composition prepared above was placed in a stainless steel fixed bed reactor. The temperature was set to 300° C. under a nitrogen flow. Isopentane was then introduced into the reactor. The system was run at a weight hourly space velocity (WHSV) of 1.9 for 9 hours. After about two hours and 23 minutes, the temperature was raised to 350° C. After four (4) hours on stream, the temperature was raised to 400° C. After about six (6) hours and twenty-two (22) minutes on-stream, the temperature was raised to 450° C. The results are shown in Table I. The abbreviation “TON” stands for Turn Over Number. This is measured on a per hour basis.

TABLE I

| | | Surface | Pore | T = 300° C. | | T = 350° C. | | T = 400° C. | | T = 450° C. | |
|---|---|---|---|---|---|---|---|---|---|---|---|
| | Ir | Area | Volume | Conv | TON | Conv. | TON | Conv. | TON | Conv. | TON |
| Example | (wt %) | (m²g) | cc/g | (%) | (/h) | (%) | (/h) | (%) | (/h) | (%) | (/h) |
| I | 0.31 | 311 | 1.12 | 2.3 | 37 | 6.2 | 95 | 14.9 | 224 | 23.0 | 295 |
| II | 0.17 | 287 | 1.61 | 2.8 | 80 | 6.6 | 194 | 14.6 | 440 | 23.1 | 602 |

### Example II

Undried silica was impregnated with 52 milligrams of hydridocarbonyltris(triphenylphosphine)iridium(I) by incipient wetness. The iridium complex was dissolved in 15.6 grams of toluene. This solution was then added drop wise to 6.002 grams of silica and was dried at about 50° C. with a purge of nitrogen.

A 4.886-gram quantity of the composition prepared above was placed in a stainless steel fixed bed reactor. The temperature was set to 300° C. under a nitrogen flow. Isopentane was then introduced into the reactor. The system was run with a weight hourly space velocity (WHSV) of 1.9 for 10 hours. After about two hours on-stream, the temperature was raised to 350° C. After four (4) hours on-stream, the temperature was raised to 400° C. After about six (6) hours on-stream, the temperature was raised to 450° C. The results are shown in Table I (above).

As is evident from Table I, both catalysts prepared in Examples I and II are active for the dehydrogenation of isopentane.

### Example III

Undried silica was impregnated with 100 milligrams of cis-dichlorobis-(triphenylphosphine)platinum(II) by incipient wetness. The platinum complex was dissolved in 17.76 grams of dichloromethane. This solution was then added drop wise to 6.003 grams of silica and was then dried.

A 5.002-gram quantity of the composition prepared above was placed in a stainless steel fixed bed reactor. The temperature was set to 300° C. under a nitrogen flow. Isopentane was then introduced into the reactor. The system was run with a weight hourly space velocity (WHSV) of 1.9 for 9 hours. After two hours on-stream, the temperature was raised to 350° C. After four (4) hours on stream, the temperature was raised to 400° C. After about six (6) hours on-stream, the temperature was raised to 450° C. The results are shown in Table II. Note that ‘selectivity’ refers to the percentage of the converted product which was converted into the desired product, in this case isopentene.

TABLE II

Temperature (° C.)

| | 300 | 350 | 400 | 450 |
|---|---|---|---|---|
| Conversion | 1.2 | 5.5 | 11.8 | 18.9 |
| (%) | | | | |
| Selectivity | 85.2 | 95.5 | 96.1 | 93.2 |
| (%) | | | | |

### Example IV

Undried silica was impregnated with 99 milligrams of hydridocarbonyltris(triphenylphosphine)rhodium(I) by incipient wetness. The rhodium complex was dissolved in 10.5 grams of toluene. This solution was then added drop wise to 6.001 grams of silica and was then dried.

A 5.007-gram quantity of the composition prepared above was placed in a stainless steel fixed bed reactor. The temperature was set to 300° C. under a nitrogen flow. Isopentane was then introduced into the reactor. The system was run with a weight hourly space velocity (WHSV) of 1.9 for 9 hours. After two hours on-stream, the temperature was raised to 350° C. After four (4) hours and forty-one (41) minutes on-stream, the temperature was raised to 400° C. After six (6) hours on-stream, the temperature was raised to 450° C. The results are shown in Table III.

TABLE III

Temperature (° C.)

| | 300 | 350 | 400 | 450 |
|---|---|---|---|---|
| Conversion | 1.2 | 5.5 | 11.8 | 18.9 |
| (%) | | | | |
| Selectivity | 85.2 | 95.5 | 96.1 | 93.2 |
| (%) | | | | |

### Example V

Undried silica was impregnated with 100 milligrams of hydridocarbonyltris(triphenylphosphine)iridium(I) by incipient wetness. The iridium complex was dissolved in 10.5 grams of toluene. This solution was then added drop wise to 6.006 grams of silica and was dried at about 50° C. and with a purge of nitrogen.

A 5.015-gram quantity of the composition prepared above was placed in a stainless steel fixed bed reactor. The temperature was set to 300° C. under a nitrogen flow. Normal pentane was then introduced into the reactor. The system was run with a weight hourly space velocity (WHSV) of 1.9 for 9 hours. After two hours on-stream, the temperature was raised to 350° C. After four (4) hours on stream, the temperature was raised to 400° C. After six (6) hours on-stream, the temperature was raised to 450° C. The results are shown in Table IV.

TABLE IV

Temperature (° C.)

| | 300 | 350 | 400 | 450 |
|---|---|---|---|---|
| Conversion | 2 | 6.4 | 10.8 | 12.5 |
| (%) | | | | |
| Selectivity | 94.4 | 94.2 | 88.5 | 81.7 |
| (%) | | | | |

### Example VI

Undried silica was impregnated with 301 milligrams of hydridocarbonyltris(triphenylphosphine)iridium(I) by incipient wetness. The iridium complex was dissolved in 31.53 grams of toluene. This solution was then added drop wise to 18.015 grams of silica and was dried at about 50° C. and with a purge of nitrogen.

A 5.004-gram quantity of the composition prepared above was placed in a stainless steel fixed bed reactor. The temperature was set to 300° C. under a nitrogen flow. Cyclopentane was then introduced into the reactor. The system was run with a weight hourly space velocity (WHSV) of 1.9 for 13 hours. After three hours on-stream, the temperature was raised to 350° C. After six (6) hours on stream, the temperature was raised to 400° C. After eight (8) hours on-stream, the temperature was raised to 450° C. The results are shown in Table V.

TABLE V

Temperature (° C.)

| | 300 | 350 | 400 | 450 |
|---|---|---|---|---|
| Conversion | 2.0 | 4.0 | 4.5 | 5.3 |
| (%) | | | | |
| Selectivity | 90 | 86.5 | 80.8 | 75.5 |
| (%) | | | | |

### Example VII

Dried silica was impregnated with 100 milligrams of (tricyclohexylphosphine)(1,5-cyclooctadiene)(pyridine)iridium(I) hexafluorophosphate by incipient wetness. The iridium complex was dissolved in 20.72 grams of dichloromethane. This solution was then added drop wise to 7 grams of silica (20×40 mesh granules), with a surface area of 315 m²/g and a pore volume of 1.1 cc/g, and was dried in a vacuum.

A 5.007-gram quantity of the composition prepared above was placed in a stainless steel fixed bed reactor. The temperature was set to 300° C. under a nitrogen flow. Isopentane was then introduced into the reactor. The system was run at a weight hourly space velocity (WHSV) of 1.9 for 9 hours. After about two hours and 10 minutes, the temperature was raised to 350° C. After five (5) hours on stream, the temperature was raised to 400° C. After about seven (7) hours and fifty (50) minutes on-stream, the temperature was raised to 450° C. The results are shown in Table VI.

### Example VIII

Undried silica was impregnated with 102 milligrams of (tricyclohexylphosphine)(1,5-cyclooctadiene)(pyridine)iridium(I) hexafluorophosphate by incipient wetness. The iridium complex was dissolved in 20.72 grams of dichloromethane. This solution was then added drop wise to 7 grams of silica and was dried at about 50° C. with a purge of nitrogen.

A 5-gram quantity of the composition prepared above was placed in a stainless steel fixed bed reactor. The temperature was heated to 300° C. under a nitrogen flow. Isopentane was then introduced into the reactor. The system was run with a weight hourly space velocity (WHSV) of 1.9 for 10 hours. After about a half hour on-stream, the temperature was raised to 350° C. After about five and one-half (5½) hours on-stream, the temperature was raised to 400° C. After about nine and one-half (9½) hours on-stream, the temperature was raised to 450° C. The results are shown in Table VI.

TABLE VI

| Example | Silica | Temperature (° C.) | 300 | 350 | 400 | 450 |
|---|---|---|---|---|---|---|
| VII | Dried | Conversion (%) | 0.5 | 0.9 | 2.3 | 4.1 |
| VIII | Undried | Conversion (%) | 1.2 | 4.4 | 8.3 | 9.6 |

As is evident from Table VI, the catalyst composition prepared with undried silica shows higher conversion than the catalyst prepared with dried silica.

### Example IX

Undried silica was impregnated with 0.84 grams of hydridocarbonyltris(triphenylphosphine)iridium(I) by incipient wetness. The iridium complex was dissolved in 26 milliliters of toluene. This solution was then added drop wise to 20 grams of silica and was dried at about 50° C. with a purge of nitrogen.

A 2.004-gram quantity of the composition prepared above was placed in a stainless steel fixed bed reactor. The temperature was set to 350° C. under a nitrogen flow, and continued at that temperature for four hours. A separate 2.011-gram quantity of the composition prepared above was placed in a stainless steel fixed bed reactor. The temperature was set to 450° C. under a nitrogen flow and was maintained at that temperature for five hours. Then, a separate 2.001-gram quantity of the composition prepared above was placed in a stainless steel fixed bed reactor. The composition was heated to 550° C. under a nitrogen flow and continued to be heated at that temperature for four hours.

The results for the composition heated to various temperatures are shown in Table VII.

TABLE VII
Example IX Composition Heated to Various Temperatures

| T | Ir | P | C | H | P/Ir | C/P |
|---|---|---|---|---|---|---|
| (° C.) | Wt % | Wt % | Wt % | Wt % | Molar Ratio | Molar Ratio |
| 350 | 0.92 | 0.26 | 0.67 | 0.81 | 1.8 | 6.6 |
| 450 | 0.86 | 0.28 | 0.26 | 0.70 | 2.0 | 2.4 |
| 550 | 0.89 | 0.27 | 0.26 | 0.54 | 1.9 | 2.5 |

### Example X

Undried silica was impregnated with 0.504 grams of hydridocarbonyltris(triphenylphosphine)iridium(I) by incipient wetness. The iridium complex was dissolved in 18 mL of toluene. The solution was then added drop wise to 12 grams of the silica and was then dried.

A 3.67-gram quantity of the composition prepared above was placed in a stainless steel fixed bed reactor along with 18.21 grams of an inert support material. The temperature was raised to 400° C. and an isopentane feed was introduced to the reactor at a rate of 18.6 mL/hour. After 44 hours on-stream, hydrogen was introduced to the reactor at a rate of 100 mL/min while the temperature was set at 260° C. One and a half hours later, the temperature was increased to 400° C. The flow of hydrogen was cut off, and nitrogen was then added to the reactor at a rate of 900 mL/hour for 30 minutes. Then, the isopentane feed was once again restarted at 18.6 mL/hour.

Then, after 67.5 hours on-stream (from the beginning), the temperature was lowered to 260° C. and the reactor was purged with nitrogen for one hour. The temperature was then raised to 390° C. under a 100-mL/minute nitrogen flow. Thirty minutes later, the flow of nitrogen was stopped. The temperature was held at 390° C. and the isopentane feed was restarted at a rate of 18.6 mL/hour.

After 71.5 hours on-stream (from the beginning) the isopentane feed was cut off and hydrogen was introduced to the reactor at a rate of 100 mL/minute, two hours and ten minutes later, the hydrogen flow was cut off and a nitrogen purge was applied at 900 mL/min for 30 minutes. Then, the isopentane feed was restarted at 18.6 mL/hour at a temperature of 390° C. After 89 total hours on-stream, the feed was cut off and the reactor was cooled down. The results for the hydrogen reactivation are shown in Table VIII.

TABLE VIII
Isopentane Conversion (%)

| | Time on | | | | | | | |
|---|---|---|---|---|---|---|---|---|
| | Stream, hrs. | | | | | | | |
| | 1 | 2 | 3.5 | 5 | 20 | 28 | 29 | 44 |
| Fresh | | | | 10.8 | 9.86 | 8.75 | 8.64 | 8.17 |
| Catalyst | | | | | | | | |
| 1^{st} H₂ | 10.6 | 11.1 | 10.1 | | 8.86 | 8.13 | | |
| Regen. | | | | | | | | |
| 2^{nd} H₂ | | | 8.21 | | 4.21 | | | |
| Regen. | | | | | | | | |

### Example XI

Undried silica was impregnated with 125 milligrams of hexachloroiridic acid by incipient wetness. The hexachloroiridic acid was dispersed in 13.2 grams of water. This solution was then added drop wise to 6.003 grams of silica. The loaded silica was then dried with a purge of nitrogen. Then, 322 milligrams of triphenylphosphine were dispersed in 7.2 grams of pentane, and added drop wise to the silica/hexachloroiridic acid composition. This was then dried at room temperature.

A 5.008-gram quantity of the composition prepared above was placed in a stainless steel fixed bed reactor. The temperature was set to 300° C. under a nitrogen flow. Isopentane was then introduced into the reactor. The system was run for ten hours. After two hours, the temperature was raised to 350° C. After four hours on stream, the temperature was raised to 400° C. After six hours on-stream, the temperature was raised to 450° C. The results are shown in Table IX.

TABLE IX

Temperature (° C.)

| | 300 | 350 | 400 | 450 |
|---|---|---|---|---|
| Conversion (%) | 2.0 | 7.1 | 16.3 | 27.4 |

### Example XII

Undried silica was impregnated with 105 milligrams of bis(1,5-cyclooctadiene)iridium(I)tetrafluoroborate by incipient wetness. The iridium compound was dispersed in 20.7 grams of dichloromethane. This solution was then added drop wise to 7.001 grams of silica and was then dried with a purge of nitrogen.

A 5.007-gram quantity of the composition prepared above was placed in a stainless steel fixed bed reactor. The temperature was set to 300° C. under a nitrogen flow. Isopentane was then introduced into the reactor. The system was run with a weight hourly space velocity (WHSV) of 1.9 for about eight hours and twelve minutes. After about one hour and forty minutes on-stream, the temperature was raised to 350° C. After about three hours and forty minutes on-stream, the temperature was raised to 400° C. After about five hours and forty minutes on-stream, the temperature was raised to 450° C. The results are shown in Table X.

TABLE X

| | Excess | | | | | | |
|---|---|---|---|---|---|---|---|
| | PPh₃ | 300° C. | | 400° C. | | 450° C. | |
| | (molar | Conv. | TON | Conv. | TON | Conv. | TON |
| Example | ratio) | (%) | (h⁻¹) | (%) | (h⁻¹) | (%) | (h⁻¹) |
| XII | 0 | 0.1 | 0.8 | 0.4 | 3 | 1.7 | 11 |
| XIII | 4x | 2.1 | 14.7 | 5.6 | 41.4 | 20.8 | 119 |
| XIV | 8x | 1.3 | 9.1 | 6.4 | 46.6 | 24.4 | 143.7 |

Note that the Example XII compound contains no triphenylphosphine, while the Examples XIII and XIV compounds contain 4 and 8 times excess triphenylphosphine, respectively.

As is evident from Table X, the compounds containing triphenylphosphine show a higher percent conversion of isopentane, especially at higher temperatures.

### Example XIII

Undried silica was impregnated with 100 milligrams of bis(cyclooctadiene)iridium(I)tetrafluoroborate by incipient wetness. The iridium compound was dispersed in 17.78 grams of dichloromethane. This solution was then added drop wise to 6.002 grams of silica and was then dried with a nitrogen stream.

Then, 212 milligrams of triphenylphosphine were dispersed in 7.2 grams of pentane, and added drop wise to the silica/iridium composition. This was then dried in a nitrogen flow.

A 5.008-gram quantity of the composition prepared above was placed in a stainless steel fixed bed reactor. The temperature was set to 300° C. under a nitrogen flow. Isopentane was then introduced into the reactor. The system was run with a weight hourly space velocity (WHSV) of 1.9 for about eight hours and twelve minutes. After about one hour and forty minutes on-stream, the temperature was raised to 350° C. After about three hours and forty minutes on stream, the temperature was raised to 400° C. After about five hours on-stream, the temperature was raised to 450° C. The results are shown in Table X.

### Example XIV

Undried silica was impregnated with 101 milligrams of bis(1,5-cyclooctadiene)iridium(I)tetrafluoroborate by incipient wetness. The iridium compound was dispersed in 17.8 grams of dichloromethane. This solution was then added drop wise to 6.010 grams of silica and was then dried with a nitrogen stream.

Then 427 milligrams of triphenylphosphine were dispersed in 7.2 grams of pentane, and added drop wise to the silica/iridium composition. This was then dried in a nitrogen flow.

A 5.003-gram quantity of the composition prepared above was placed in a stainless steel fixed bed reactor. The temperature was set to 300° C. under a nitrogen flow. Isopentane was then introduced into the reactor. The system was run for a total of nine hours. After two hours on-stream, the temperature was raised to 350° C. After four hours on-stream, the temperature was raised to 400° C. After six hours on-stream, the temperature was raised to 450° C. The results are shown in Table X (above).

### Example XV

Undried silica was impregnated with 120 milligrams of NiCl₂ by incipient wetness. The NiCl₂ was added to 13.2 grams of water. This solution was then added drop wise to 6.018 grams of silica. The loaded silica was then dried with gentle heat and with a purge of nitrogen.

Then 0.97 grams of triphenylphosphine were dispersed in 7.2 grams of pentane, and added drop wise to the silica/nickel composition. This composition was then dried.

A 5-gram quantity of the composition prepared above was placed in a stainless steel fixed bed reactor. The temperature was set to 300° C. under a nitrogen flow. Isopentane was then introduced into the reactor. The system was run for about eight hours. After about two hours on-stream, the temperature was raised to 350° C. After about four hours and ten minutes on stream, the temperature was raised to 400° C. After six hours on-stream, the temperature was raised to 450° C. The results are shown in Table XI (below).

### Example XVI

Dried silica was impregnated with 0.44 grams of nickel(II)acetylacetonate by incipient wetness. The nickel compound was dispersed in 29.8 grams of dichloromethane. This solution was then added drop wise to 10 grams of silica and was then dried in a vacuum.

A 5.006-gram quantity of the composition prepared above was placed in a stainless steel fixed bed reactor. The temperature was set to 300° C. under a nitrogen flow. Isopentane was then introduced into the reactor. The system was run for ten hours. After three hours on-stream, the temperature was raised to 400° C. After six hours on stream, the temperature was raised to 500° C. The results are shown in Table XI.

TABLE XI

| | 300° C. | | 350° C. | | 400° C. | | 450° C. | |
|---|---|---|---|---|---|---|---|---|
| | | TON | | TON | | TON | | TON |
| Example | Conv. (%) | (h⁻¹) | Conv. (%) | (h⁻¹) | Conv. (%) | (h⁻¹) | Conv. (%) | (h⁻¹) |
| XV | 1.69 | 3.1 | 0.03 | 0.1 | 0 | 0 | 0.07 | 0.1 |
| XVI | 1.4 | 3.5 | 0.02 | 0.1 | 0.1 | 0.2 | 0.3 | 0.5 |

As is evident from Table XI, both nickel-containing compounds are active for the conversion of isopentane to isopentenes.

### Example XVII

Undried silica was impregnated with 68 milligrams of hexachloroiridic acid by incipient wetness. The hexachloroiridic acid was dispersed in 13.2 grams of water. This solution was then added drop wise to 6.005 grams of silica. The loaded silica was then dried with heat and a purge of nitrogen. Then, 175 milligrams of triphenylphosphine were dispersed in 7.2 grams of pentane, and added drop wise to the silica/iridium composition. This was then dried at room temperature.

A 5.002-gram quantity of the composition prepared above was placed in a stainless steel fixed bed reactor. The temperature was set to 300° C. under a nitrogen flow. Isopentane was then introduced into the reactor. The system was run nine hours. After two hours, the temperature was raised to 350° C. After four hours on stream, the temperature was raised to 400° C. After six hours on-stream, the temperature was raised to 450° C. The results are shown in Table XII (below).

As is evident from Table XII, silica compounds with various amounts of platinum and iridium are effective for converting isopentane to isopentenes. Compounds containing more iridium are more effective.

### Example XVIII

Undried silica was impregnated with 119 milligrams of hexachloroplatinic acid by incipient wetness. The hexachloroplatinic acid was dispersed in 13.2 grams of water. This solution was then added drop wise to 6.040 grams of silica and was then dried with a purge of nitrogen. Then 305 milligrams of triphenylphosphine were dispersed in 7.2 grams of pentane, and added drop wise to the silica/platinum composition. This was then dried at room temperature.

A 5.006-gram quantity of the composition prepared above was placed in a stainless steel fixed bed reactor. The temperature was set to 300° C. under a nitrogen flow. Isopentane was then introduced into the reactor. The system was run for ten hours. After two hours on-stream, the temperature was raised to 350° C. After four hours on-stream, the temperature was raised to 400° C. After six hours on-stream, the temperature was raised to 450° C. After eight hours on-stream, the temperature was raised to 500° C. The results are shown in Table XII (below).

### Example XIX

Undried silica was impregnated with 51 milligrams of hexachloroiridic acid and 13 milligrams of hexachloroplatinic acid by incipient wetness. The two acids were dispersed in 13.2 grams of water. This solution was then added drop wise to 6.006 grams of silica and was then dried with a nitrogen stream and heat.

Then 164 milligrams of triphenylphosphine were dispersed in 7.2 grams of pentane, and added drop wise to the silica/iridium/platinum composition. This was then dried with a nitrogen flow.

A 5.001-gram quantity of the composition prepared above was placed in a stainless steel fixed bed reactor. The temperature was set to 300° C. under a nitrogen flow. Isopentane was then introduced into the reactor. The system was run for ten hours. After about two hours and fourteen minutes on-stream, the temperature was raised to 350° C. After about four hours on-stream, the temperature was raised to 400° C. After about seven hours on-stream, the temperature was raised to 450° C. The results are shown in Table XII (below).

### Example XX

Undried silica was impregnated with 13 milligrams of hexachloroiridic acid and 50.4 milligrams of hexachloroplatinic acid by incipient wetness. The two acids were dispersed in 13.2 grams of water. This solution was then added drop wise to 6 grams of silica. The loaded silica was then dried with a nitrogen stream and heat.

Then 164 milligrams of triphenylphosphine were dispersed in 7.2 grams of pentane, and added drop wise to the silica/iridium/platinum composition. This was then dried with a nitrogen flow.

A 5.006-gram quantity of the composition prepared above was placed in a stainless steel fixed bed reactor. The temperature was set to 300° C. under a nitrogen flow. Isopentane was then introduced into the reactor. The system was run for a total of ten hours. After two hours on-stream, the temperature was raised to 350° C. After four hours and forty minutes on-stream, the temperature was raised to 400° C. After seven hours on-stream, the temperature was raised to 450° C. The results are shown in Table XII (below).

### Example XXI

Undried silica was impregnated with 32 milligrams of hexachloroiridic acid and 32 milligrams of hexachloroplatinic acid by incipient wetness. The two acids were dispersed in 13.2 grams of water. This solution was then added drop wise to 6 grams of silica. The loaded silica was then dried with heat and with a purge of nitrogen.

Then 163 milligrams of triphenylphosphine were dispersed in 7.2 grams of pentane, and added drop wise to the silica/iridium/platinum composition. This composition was then dried with a nitrogen flow.

A 5-gram quantity of the composition prepared above was placed in a stainless steel fixed bed reactor. The temperature was set to 300° C. under a nitrogen flow. Isopentane was then introduced into the reactor. The system was run for about nine hours and twelve minutes. After two hours on-stream, the temperature was raised to 350° C. After about four hours and twenty-two minutes on stream, the temperature was raised to 400° C. After about six hours and fifteen minutes on-stream, the temperature was raised to 450° C. The results are shown in Table XII (below).

TABLE XII

| | Ir:Pt Wt. | Conversion (%) | | | |
|---|---|---|---|---|---|
| Example | Ratio | 300° C. | 350° C. | 400° C. | 450° C. |
| XVII | No Pt | 0.7 | 4.4 | 11 | 18.6 |
| XVIII | No Ir | 0.1 | 1.4 | 4.5 | 6.5 |
| XIX | 4:1 | 1.2 | 7.5 | 15.2 | 23.7 |
| XX | .25:1 | 0.3 | 2.8 | 7.4 | 9.9 |
| XXI | 1:1 | 0.8 | 5.9 | 12.6 | 18.4 |

### Example XXII

Undried silica was impregnated with 157 milligrams of hexachloroplatinic acid by incipient wetness. The acid was dispersed in 7.7 grams of water. This solution was then added drop wise to 7.010 grams of silica and was dried in a vacuum.

A 5.003-gram quantity of the composition prepared above was placed in a stainless steel fixed bed reactor. The temperature was set to 300° C. under a nitrogen flow. Isopentane was then introduced into the reactor. The system was run for about ten hours and eight minutes. After three hours on-stream, the temperature was raised to 350° C. After five hours on stream, the temperature was raised to 400° C. After seven hours on-stream, the temperature was raised to 450° C. The results comparing the composition in Example XXII to that of Example XVIII, are shown in Table XIII.

TABLE XIII

| | | 300° C. | | 350° C. | | 400° C. | | 450° C. | |
|---|---|---|---|---|---|---|---|---|---|
| | Amount | Conv. | TON | Conv. | TON | Conv. | TON | Conv. | TON |
| Example | of PPh₃ | (%) | (h⁻¹) | (%) | (h⁻¹) | (%) | (h⁻¹) | (%) | (h⁻¹) |
| XXII | none | 0.1 | 0.4 | 0.2 | 0.7 | 0.3 | 1.1 | 0.4 | 2.3 |
| XVIII | 4x | 0.2 | 0.1 | 0.4 | 1.0 | 5.1 | 26.7 | 7.7 | 36.4 |
| | excess | | | | | | | | |

### Example XXIII

Silica was impregnated with 1.033 grams of bis(1,4-cyclooctadiene)iridium(I)tetrafluoroborate by incipient wetness. The iridium compound was dispersed in 50 milliliters of dichloromethane. This solution was then added drop wise to 40 grams of silica and the resulting compound was dried with a purge of nitrogen. Then 4.36 grams of triphenylphosphine was dispersed in 60 milliliters of dichloromethane. This solution was added drop wise to the iridium/silica material. The resulting composition was then dried.

A 2.072-gram quantity of the composition prepared above was placed in a stainless steel fixed bed reactor. The temperature was set to 450° C. under a nitrogen flow, and continued at that temperature for six hours. A separate 2.015-gram quantity of the composition prepared above was placed in a stainless steel fixed bed reactor. The temperature was set to 550° C. under a nitrogen flow and continued at that temperature for four hours.

The results for the elemental composition after being heated to various temperatures are shown in Table XIV.

TABLE XIV
Example XXIII Composition Heated to Various Temperatures

| T | Ir | P | C | H | P/Ir | C/P |
|---|---|---|---|---|---|---|
| (° C.) | Wt % | Wt % | Wt % | Wt % | Molar Ratio | Molar Ratio |
| 450 | 1.03 | 0.35 | 0.72 | 0.35 | 2.1 | 5.3 |
| 550 | 1.04 | 0.34 | 0.40 | 0.59 | 2.0 | 3.0 |

### Example XXIV

A sample of the composition prepared in Example XXIII was charged to a reactor and run at the indicated temperatures to dehydrogenate isopentane. After a 6 hour run at each temperature, the catalysts were removed and analyzed for the indicated elements.

The results are shown in Table XV.

TABLE XV
Ex. XXIII Composition with Isopentane Heated to Various
Temperatures

| | Ir | P | C | H | P/Ir | C/P |
|---|---|---|---|---|---|---|
| T (° C.) | (wt %) | (wt %) | (wt %) | (wt %) | mole ratio | mole ratio |
| 20 | 0.93 | 0.93 | 8.71 | 1.14 | 6.2 | 24.2 |
| 150 | 0.92 | 0.79 | 7.45 | 0.99 | 5.3 | 24.3 |
| 250 | 0.92 | 0.46 | 3.41 | 0.77 | 3.1 | 19.1 |
| 350 | 0.94 | 0.52 | 1.68 | 0.59 | 3.4 | 8.3 |
| 450 | 1.02 | 0.57 | 1.21 | 0.36 | 3.5 | 5.5 |
| 550 | 0.98 | 0.39 | 4.22 | 0.42 | 2.5 | 28.6 |

As is evident from Table XV, as the temperature is increased to 550° C., the level of carbon increases significantly. This is almost certainly due to coking of the feedstock.

### Example XXV

Silicon carbide was impregnated with 6.26 grams of hydridocarbonyltris(triphenylphosphine)iridium(I) by incipient wetness. The iridium complex was dispersed in 12 mL of toluene. This solution was then added drop wise to 6.044 grams of silicon carbide. This was then dried in a vacuum.

A 5.059-gram quantity of the composition prepared above was placed in a stainless steel fixed bed reactor. The temperature was set to 450° C. under a nitrogen flow. A feed of isopentane was then introduced into the reactor. The system was run seven hours and was then shut down. The results are shown in Table XVI.

TABLE XVI

| | Time On- | | | | | | |
|---|---|---|---|---|---|---|---|
| | stream, hrs. | | | | | | |
| | 1 | 2 | 3 | 4 | 5 | 6 | 7 |
| Conversion (%) | 22.30 | 23.35 | 20.69 | 19.36 | 18.45 | 17.06 | 16.98 |

### Example XXVI

Silicon nitride was impregnated with 0.267 grams of hydridocarbonyltris(triphenylphosphine)iridium(I) by incipient wetness. The iridium complex was dispersed in 12 mL of toluene. This solution was then added drop wise to 6.004 grams of silicon nitride. This was then dried in a vacuum.

A 5.031-gram quantity of the composition prepared above was placed in a stainless steel fixed bed reactor. The temperature was set to 450° C. under a nitrogen flow. A feed of isopentane was then introduced into the reactor. The system was run 9 hours and 19 minutes before being shut down. The results are shown in Table XVI.

TABLE XVII

| | Time On- | | | | | | | | |
|---|---|---|---|---|---|---|---|---|---|
| | stream, hrs. | | | | | | | | |
| | 1 | 2 | 3 | 4.33 | 5.33 | 6.33 | 7.33 | 8.33 | 9.33 |
| Conver- | 21.83 | 22.61 | 21.73 | 20.81 | 20.92 | 19.09 | 18.64 | 19.41 | 17.58 |
| sion | | | | | | | | | |
| (%) | | | | | | | | | |

While this invention has been described in detail for the purpose of illustration, it should not be construed as limited thereby, but intended to cover all changes and modifications within the spirit and scope thereof.

What is claimed is:

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. A process comprising:
contacting a hydrocarbon feed with a catalyst in a dehydrogenation reaction zone under dehydrogenation reaction conditions wherein said catalyst at a temperature range of from about 0° C. to about 400° C. comprises:
a) a complex comprising
i) at least one metal selected from the group consisting of Group 8 metals, Group 9 metals, Group 10 metals, and combinations thereof; and
ii) a compound having the formula R₃X wherein R is selected from the group consisting of hydrogen, an alkyl, an alkenyl, an alkynyl, cycloalkyls, cycloalkenyls, cycloalkyls, aryls, substituted organic compounds and X is a Group 15 element selected from the group consisting of nitrogen, phosphorus, arsenic, antimony and bismuth; and
b) a support component comprising a silicon-containing compound.

54. A process in accordance with claim 53 wherein said at least one metal is selected from the group consisting of iridium, rhodium, platinum, nickel, cobalt, palladium, iron, ruthenium, osmium, and combinations of two or more thereof.

55. A process in accordance with claim 53 wherein R is a phenyl group.

56. A process in accordance with claim 53 wherein R is a cyclohexyl group.

57. A process in accordance with claim 53 wherein said organophosphine is a triphenylphosphine.

58. A process in accordance with claim 53 wherein said organophosphine is a tricyclohexylphosphine.

59. A process in accordance with claim 53 wherein said at least one metal is iridium.

60. A process in accordance with claim 53 wherein said at least one metal is rhodium.

61. A process in accordance with claim 53 wherein said at least one metal is platinum.

62. A process in accordance with claim 53 wherein said at least one metal is nickel.

63. A process in accordance with claim 53 wherein X is phosphorus.

64. A process in accordance with claim 53 wherein said at least one metal is present in said catalyst in a weight percent in the range of from about 0.01 to about 10 weight percent based on the total weight of said catalyst.

65. A process in accordance with claim 53 wherein said at least one metal is present in said catalyst in a weight percent in the range of from about 0.1 to about 5 weight percent based on the total weight of said catalyst.

66. A process in accordance with claim 53 wherein said at least one metal is present in said catalyst in a weight percent in the range of from about 0.2 to about 2.0 weight percent based on the total weight of said catalyst.

67. A process in accordance with claim 53 wherein said catalyst is reactivated by stripping with hydrogen.

68. A process in accordance with claim 53 wherein said dehydrogenation reaction conditions comprise a reaction temperature in the range of from about 150° C. to about 1000° C.

69. A process in accordance with claim 53 wherein said dehydrogenation reaction conditions comprise a reaction temperature in the range of from about 200° C. to about 650° C.

70. A process in accordance with claim 53 wherein said dehydrogenation reaction conditions comprise a reaction temperature in the range of from about 300° C. to about 650° C.

71. A process in accordance with claim 53 wherein said silicon-containing compound is silica.

72. A process in accordance with claim 53 wherein said silicon-containing compound is silicon carbide.

73. A process in accordance with claim 53 wherein said silicon-containing compound is silicon nitride.

74. A process in accordance with claim 53 wherein said hydrocarbon feed comprises hydrocarbons having in the range of from 2 to 10 carbon atoms per molecule.

75. A process in accordance with claim 53 wherein said hydrocarbon feed is selected from the group consisting of normal pentane, isopentane, cyclopentane, and combinations of two or more thereof.

76. A process in accordance with claim 53 wherein said hydrocarbon feed comprises isopentane.

77. A process in accordance with claim 53 wherein said catalyst is prepared by a method comprising:
a) admixing
1) a liquid and
2) a complex comprising
i) a compound having the formula R₃X wherein R is selected from the group consisting of hydrogen, an alkyl, an alkenyl, an alkynyl, cycloalkyls, cycloalkenyls, cycloalkynyls, aryls, substituted aryls, and substituted organic compounds and X is a Group 15 element selected from the group consisting of nitrogen, phosphorus, arsenic, antimony and bismuth; and
ii) at least one metal selected from the group consisting of Group 8 metals, Group 9 metals, Group 10 metals, and combinations thereof to form a mixture thereof, and
b) incorporating said mixture into or onto a silicon-containing compound.

78. A process in accordance with claim 77 wherein R is a phenyl group.

79. A process in accordance with claim 77 wherein R is cyclohexyl group.

80. A process in accordance with claim 77 wherein X is phosphorus.

81. A process in accordance with claim 77 wherein said compound having the formula R₃X is triphenylphosphine.

82. A process in accordance with claim 77 wherein said compound having the formula R₃X is tricyclohexylphosphine.

83. A process in accordance with claim 77 wherein said at least one metal is iridium.

84. A process in accordance with claim 77 wherein said at least one metal is rhodium.

85. A process in accordance with claim 77 wherein said at least one metal is platinum.

86. A process in accordance with claim 77 wherein said silicon-containing compound has a pore volume in the range of from about 0.01 cm³/g to about 10 cm³/g.

87. A process in accordance with claim 77 wherein said silicon-containing compound has a surface area in the range of from about 1 m²/g to about 1000 m²/g.

88. A process in accordance with claim 77 wherein said silicon-containing compound is undried.

89. A process in accordance with claim 77 wherein said silicon-containing compound is silica.

90. A process in accordance with claim 77 wherein said silicon-containing compound is silicon carbide.

91. A process in accordance with claim 77 wherein said silicon-containing compound is silicon nitride.

92. A process in accordance with claim 53 wherein said catalyst is prepared by a method comprising:
a) incorporating a compound comprising at least one metal selected from the group consisting of Group 8 metals, Group 9 metals, Group 10 metals, and combinations thereof into or onto a silicon-containing compound to form a first incorporated mixture;
b) drying said first incorporated mixture to form a dried first incorporated mixture;
c) incorporating a compound having the formula R₃X, wherein R is selected from the group consisting of hydrogen, an alkyl, an alkenyl, an alkynyl, cycloalkyls, cycloalkenyls, cycloalkynyls, aryls, substituted aryls, and substituted cyclic organic compounds and X is a Group 15 element selected from the group consisting of nitrogen, phosphorus, arsenic, antimony, and bismuth; into or onto said dried first incorporated mixture so as to form a second incorporated mixture; and
d) drying said second incorporated mixture to form said catalyst.

93. A process in accordance with claim 92 wherein R is a phenyl group.

94. A process in accordance with claim 92 wherein R is a tricyclohexyl group.

95. A process in accordance with claim 92 wherein X is phosphorus.

96. A process in accordance with claim 92 wherein said at least one metal is iridium.

97. A process in accordance with claim 92 wherein said at least one metal is rhodium.

98. A process in accordance with claim 92 wherein said at least one metal is platinum.

99. A process in accordance with claim 92 wherein said at least one metal is nickel.

100. A process in accordance with claim 99 wherein said compound comprising at least one metal and said compound having the formula R₃X are incorporated into or onto said silicon-containing compound simultaneously.

101. A process in accordance with claim 92 wherein said silicon-containing compound has a pore volume in the range of from about 0.1 cm³/g to about 10 cm³/g.

102. A process in accordance with claim 92 wherein said silicon-containing compound has a surface area in the range of from about 50 m²/g to about 600 m²/g.

103. A process in accordance with claim 92 wherein said silicon-containing compound is silica.

104. A process in accordance with claim 92 wherein said silicon-containing compound is silicon carbide.

105. A process in accordance with claim 92 wherein said silicon-containing compound is silicon nitride.

106. A process in accordance with claim 92 wherein said drying in steps b) and d) occurs at a temperature in the range of from about 15° C. to about 120° C.

107. A process in accordance with claim 92 wherein said silicon-containing compound is undried.
